(19) United States
(12) Patent Application Publication
(10) Pub. No.: US 2017/0065827 A1
(43) Pub. Date: Mar. 9, 2017

(54) LIGHTING CONTROL SYSTEM

(71) Applicant: PANASONIC INTELLECTUAL PROPERTY MANAGEMENT CO., LTD., Osaka (JP)

(72) Inventors: Yuri FUJIWARA, Osaka (JP); Ayako TSUKITANI, Osaka (JP); Naohiro TODA, Chiba (JP)

(21) Appl. No.: 15/258,596

(22) Filed: Sep. 7, 2016

(30) Foreign Application Priority Data
Sep. 8, 2015 (JP) 2015-176996

(51) Int. Cl. A61N 5/06; A61B 10/00; H05B 37/02

(52) U.S. Cl. CPC A61N 5/0618; H05B 37/0227; H05B 37/0272; A61B 10/0012; A61N 2005/0626; A61B 2010/0019; A61B 2010/0029

(57) ABSTRACT

A lighting control system includes: a biological information obtainer which obtains biological information related to a biological body of a user; an environmental information obtainer which obtains environmental information related to a surrounding environment of the user; a severity determiner which determines a severity of premenstrual syndrome (PMS) based on the biological information obtained by the biological information obtainer and the environmental information obtained by the environmental information obtainer; and a lighting controller which controls a lighting device based on the severity determined by the severity determiner.

### CROSS REFERENCE TO RELATED APPLICATION

This application claims the benefit of priority of Japanese Patent Application Number 2015-176996 filed on Sep. 8, 2015, the entire content of which is hereby incorporated by reference.

### BACKGROUND

1. Technical Field

The present disclosure relates to a lighting control system corresponding to biological information and environmental information.

2. Description of the Related Art

A device which estimates a change in a physical condition of a user which appears in a menstrual cycle of the user, based on biological information such as a basal body temperature, and controls a temperature environment in a bed according to the estimated physical condition of the user is known. For example, Japanese Unexamined Patent Application Publication No. 2003-164527 discloses a technique related to such a device.

### SUMMARY

In the menstrual cycle, there is a period of developing premenstrual syndrome (PMS). It is known that the symptoms of PMS are alleviated as a result of receiving sunlight or illuminating light.

The technique disclosed by Japanese Unexamined Patent Application Publication No. 2003-164527, however, is to control only the temperature environment in a bed, and not targeting a lighting environment. In addition, during the period of developing PMS, the degree of the symptoms of PMS varies between severe and mild, depending on the amount of activity of a user, a period of time during which a user is exposed to sunlight or illuminating light, etc. For example, the symptoms of PMS become severe when a user does a great amount of activity, or become mild when a user is exposed to sunlight or illuminating light for a long period of time. With the technique disclosed by Japanese Unexamined Patent Application Publication No. 2003-164527, control according to variation in the severity of PMS as described above is not achieved. For instance, there are cases where it is not possible to sufficiently alleviate the symptoms of PMS even when the symptoms of PMS are severe.

In view of the above, the present disclosure provides a lighting control system capable of alleviating the symptoms of PMS according to the severity of PMS.

A lighting control system according to an aspect of the present disclosure includes: a biological information obtainer which obtains biological information related to a biological body of a user; an environmental information obtainer which obtains environmental information related to a surrounding environment of the user; a severity determiner which determines a severity of premenstrual syndrome (PMS) based on the biological information obtained by the biological information obtainer and the environmental information obtained by the environmental information obtainer; and a lighting controller which controls a lighting device based on the severity determined by the severity determiner.

A lighting control system according to another aspect of the present disclosure includes: a biological information obtainer which obtains biological information related to a biological body of a user; an environmental information obtainer which obtains environmental information related to a surrounding environment of the user; a severity determiner which determines a severity of premenstrual syndrome (PMS) based on the biological information obtained by the biological information obtainer; and a lighting controller which controls a lighting device based on the environmental information obtained by the environmental information obtainer and the severity determined by the severity determiner.

With the lighting control system according to the present disclosure, it is possible to alleviate the symptoms of PMS according to the severity of PMS.

### BRIEF DESCRIPTION OF DRAWINGS

The figures depict one or more implementations in accordance with the present teaching, by way of examples only, not by way of limitations. In the figures, like reference numerals refer to the same or similar elements.

FIG. 1 is a diagram illustrating a period of developing PMS;

FIG. 2 is a diagram illustrating physical and mental conditions of women according to a menstrual cycle;

FIG. 3 is a diagram illustrating a configuration of a lighting control system according to Embodiment 1;

FIG. 4 is a flowchart illustrating an operation of the lighting control system according to Embodiment 1;

FIG. 5 is a diagram illustrating an application example of the lighting control system according to Embodiment 1;

FIG. 6 is a diagram illustrating a configuration of a lighting control system according to Embodiment 2;

FIG. 7 is a flowchart illustrating an operation of the lighting control system according to Embodiment 2; and

FIG. 8 is a diagram illustrating a configuration of a lighting control system according to another embodiment.

### DETAILED DESCRIPTION OF THE EMBODIMENTS

### Underlying Knowledge Forming the Basis of the Present Invention

First, PMS shall be described.

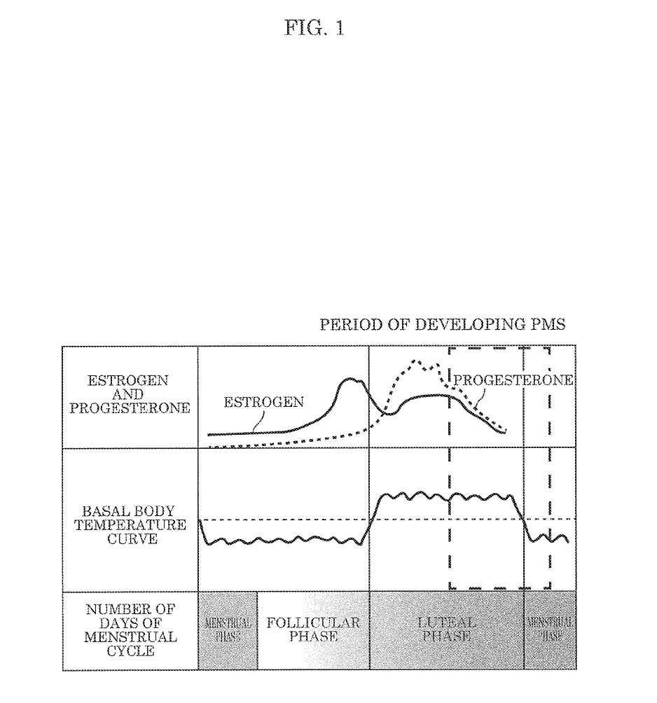

FIG. 1 is a diagram illustrating a period of developing PMS.

As illustrated in FIG. 1, the period enclosed by a dashed line is the period of developing PMS. More specifically, some women experience symptoms of PMS during a period from the latter half of the luteal phase to three or four days after the menstruation onset. It is said that PMS is related to decrease in progesterone (luteohormone).

PMS is thought to be caused, for example, by decrease in serotonin due to decrease in progesterone. Serotonin is an intracerebral substance which creates feelings of well-being and satisfaction, and stabilizes a mental state. Decrease in serotonin is likely to cause an unstable mental state. Furthermore, decrease in serotonin is likely to cause drowsiness or a problem with falling asleep, and thus likely to cause an unstable physical condition. As described above, PMS makes physical and mental conditions of women unstable.

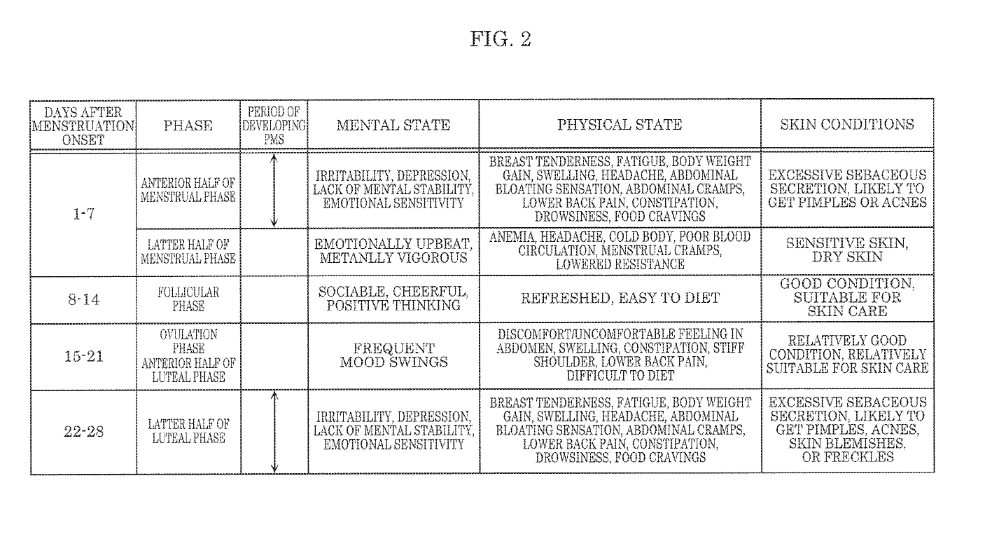

FIG. 2 is a diagram illustrating physical and mental conditions of women according to a menstrual cycle.

As illustrated in FIG. 1, PMS is developed during a period from the latter half of the luteal phase to the anterior half of the menstrual phase. Specific symptoms of PMS shall be described here. During the period of developing PMS, the mental state deteriorates by experiencing irritability, depression, lack of mental stability, emotional sensitivity, etc. Furthermore, during the period of developing PMS, the physical state deteriorates by experiencing breast tenderness, fatigue, body weight gain, swelling, headache, abdominal bloating sensation, abdominal cramps, lower back pain, constipation, drowsiness, food cravings, etc. In addition, during the period of developing PMS in the anterior half of the menstrual phase, it is likely to get pimples or acnes due to excessive sebaceous secretion, leading to a deteriorated skin condition. During the period of developing PMS in the latter half of the luteal phase, it is likely to further get skin blemishes or freckles. In such a manner, the physical and mental states and the skin condition deteriorate during the period of developing PMS. The physical and mental states and the skin condition start to improve in the latter half of the menstrual phase, become the best in the follicular phase, and start to deteriorate in the ovulation phase (the anterior half of the luteal phase). In other words, PMS develops when the physical and mental states and the skin condition deteriorate, and thus the severity of PMS can be estimated according to the physical and mental states and the skin condition.

As described above, one of causal factors of PMS is thought to be the decrease in serotonin due to decrease in progesterone, however, the decrease in serotonin can be suppressed when a user is exposed to sunlight or illuminating light during daytime. Furthermore, serotonin is secreted during daytime as a result of secretion of melatonin related to serotonin during night-time. More specifically, the rhythm of serotonin secretion of the user is adjusted by being exposed to sunlight or illuminating light during daytime, leading to alleviation of the symptoms of PMS.

As described above, the symptoms of PMS correspond to the physical and mental states and the skin condition of a user, and are alleviated according to sunlight or illuminating light to which the user is exposed. In other words, the current severity of PMS of the user is estimated according to the physical and mental states and the skin condition of the user and the sunlight or illuminating light to which the user is exposed. In addition, it is possible to alleviate the symptoms of PMS by controlling a lighting device according to the estimated severity of PMS.

The following describes in detail embodiments according to the present disclosure, with reference to the drawings. It should be noted that each of the embodiments explained below describes a specific example of the present disclosure. The numerical values, shapes, structural components, the disposition and connection of the structural components, steps, the processing order of the steps, etc. described in the following embodiments are mere examples, and do not intend to limit the present disclosure. Furthermore, among the structural components in the following embodiments, structural components not recited in any one of the independent claims which indicate the broadest concepts of the present disclosure are described as arbitrary structural components. In addition, each diagram is a schematic diagram and not necessarily strictly illustrated.

### Embodiment 1

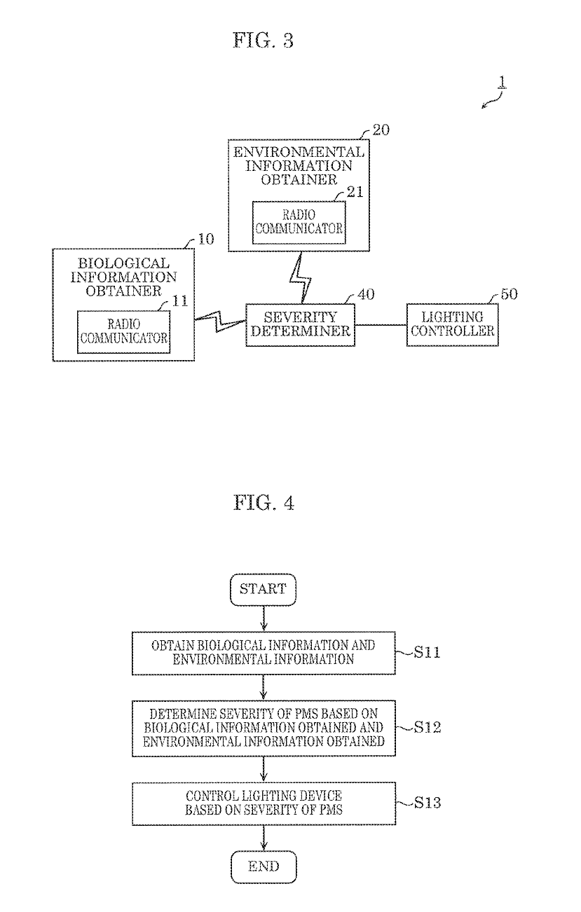

The following describes Embodiment 1 with reference to FIG. 3 to FIG. 5.

[Configuration of Lighting Control System]

FIG. 3 is a diagram illustrating a configuration of lighting control system 1 according to Embodiment 1.

Lighting control system 1 controls a lighting device according to biological information related to a biological body of a user and environmental information related to a surrounding environment of the user, and includes biological information obtainer 10, environmental information obtainer 20, severity determiner 40, and lighting controller 50.

Biological information obtainer 10 obtains biological information related to a biological body of the user. Biological information includes at least one of information items indicating a basal body temperature, an amount of luteinizing hormone secretion, an amount of luteohormone secretion, an amount of estrogenic hormone secretion, an amount of activity, blinking, electrodermal activity, brain waves, a skin condition, a date of menstruation onset, and the number of days of a menstrual cycle of the user. Biological information obtainer 10 includes radio communicator 11 for wirelessly transmitting the biological information. According to the present embodiment, radio communicator 11 wirelessly transmits the biological information to severity determiner 40.

Environmental information obtainer 20 obtains environmental information related to a surrounding environment of the user. The environmental information includes at least one of information items which indicate an amount of sunlight the user is exposed to and an illuminance of light the user is exposed to. Environmental information obtainer 20 includes radio communicator 21 for wirelessly transmitting the environmental information. According to the present embodiment, radio communicator 21 wirelessly transmits the environmental information to severity determiner 40.

Severity determiner 40 determines a severity of premenstrual syndrome (PMS) based on the biological information obtained by biological information obtainer 10 and the environmental information obtained by environmental information obtainer 20. More specifically, severity determiner 40 determines the severity of PMS based on the biological information and the environmental information each of which is quantified according to a predetermined degree of influence on the severity of PMS.

Lighting controller 50 controls a lighting device (not illustrated) based on the severity of PMS determined by severity determiner 40.

The following describes in detail operations of biological information obtainer 10, environmental information obtainer 20, severity determiner 40, and lighting controller 50, with reference to FIG. 4.

[Operation of Lighting Control System]

FIG. 4 is a flowchart illustrating an operation of lighting control system 1 according to Embodiment 1.

First, biological information obtainer 10 obtains biological information, and environmental information obtainer 20 obtains environmental information (Step S11).

Biological information obtainer 10 is, for example, a sensor for detecting the above-described biological information. Biological information obtainer 10 is, for example, an activity meter, and detects and obtains an amount of activity of the user. It should be noted that biological information obtainer 10 may be a smartphone or the like capable of obtaining the amount of activity of a user. Furthermore, biological information obtainer 10 is, for example, an electroencephalograph, and detects and obtains brain waves of a user. In addition, biological information obtainer 10 is, for example, a camera, and detects and obtains blinking, a level of bad skin condition, etc. Moreover, biological information obtainer 10 may be, for example, an input device to which the above-described biological information is provided. Biological information obtainer 10 is, for example, a personal computer (PC) or a smartphone. Biological information obtainer 10 obtains biological information as a result of a user's input of the information detected by the activity meter, the electroencephalograph, the camera, etc. Furthermore, biological information obtainer 10 obtains biological information as a result of a user's input of the information of the user's: basal body temperature; an amount of luteinizing hormone secretion; an amount of luteohormone secretion; an amount of estrogenic hormone secretion; a date of menstruation onset; the number of days of a menstrual cycle; the number of taking of a medicine for headache; etc. As described above, biological information obtainer 10 is a sensor such as an activity meter, an electroencephalograph, and a camera, or an input device such as a PC and a smartphone, and obtains biological information as a result of detection or input of the biological information. It should be noted that biological information obtainer 10 is not limited to the above-described examples, and any form can be employed as biological information obtainer 10 as long as biological information obtainer 10 is capable of obtaining biological information.

Environmental information obtainer 20 is, for example, a sensor (illuminance sensor) for detecting the above-described environmental information. Environmental information obtainer 20 is, for example, an illuminance meter, detects an amount of sunlight the user is exposed to or an illuminance of light the user is exposed to, and obtains an illuminance exposure integrated value. The illuminance exposure integrated value is a value resulting from integrating the illuminance of light the user is exposed to over a predetermined period of time. It should be noted that environmental information obtainer 20 may be, for example, a wearable terminal capable of detecting the amount of sunlight the user is exposed to or the illuminance of light the user is exposed to. In addition, environmental information obtainer 20 may be, for example, an input device to which the amount of sunlight the user is exposed to or the illuminance of light the user is exposed to is provided. Environmental information obtainer 20 is, for example, a PC or a smartphone. Environmental information obtainer 20 obtains environmental information as a result of a user's input of information detected by an illuminance meter or the like. As described above, environmental information obtainer 20 is an illuminance meter or a wearable terminal, or an input device such as a PC and a smartphone, and obtains environmental information as a result of detection or input of the environmental information. It should be noted that environmental information obtainer 20 is not limited to the above-described examples, and any form can be employed as environmental information obtainer 20 as long as environmental information obtainer 20 is capable of obtaining environmental information.

Next, severity determiner 40 determines a severity of PMS based on the biological information obtained by biological information obtainer 10 and the environmental information obtained by environmental information obtainer 20 (Step S12). The following describes a relationship between the symptoms of PMS and information items included in the biological information and the environmental information.

It is possible to detect a current phase of the user's menstrual cycle, based on the basal body temperature, the amount of luteinizing hormone secretion, the amount of luteohormone secretion, the amount of estrogenic hormone secretion, the date of menstruation onset, or the number of days of the menstrual cycle. The symptoms of PMS is severe when the user is in a period of developing PMS (i.e., a period from the latter half of the luteal phase to the anterior half of the menstrual phase), and mild when the user is in a period of not developing PMS (i.e., a period from the latter half of the menstrual phase and the anterior half of the luteal phase).

The number of taking a medicine for headache indicates the severity of headache of the user. Severe headache indicates that the symptom of PMS is severe.

The amount of activity during sleep indicates a sleep length of the user. A short sleep length indicates that the symptom of PMS is severe. The amount of activity during daytime indicates tiredness or fatigue of the user. Severe tiredness or fatigue indicates that the symptom of PMS is severe.

The brain waves indicate the number and a time length of wakes after sleep onset, a time length from getting in bed to sleep onset, and a time length from awaking to getting out of bed. A large number and a large time length of wakes after sleep indicate that the symptom of PMS is severe. In addition, a large time length from getting in bed to sleep onset, and a large time length from awaking to getting out of bed indicate that the symptom of PMS is severe.

The blinking indicates drowsiness of the user. Severe drowsiness indicates that the symptom of PMS is severe.

The level of bad skin condition indicates the number of pimples or acnes of the user. A large number of pimples or acnes indicate that the symptom of PMS is severe.

It should be noted that, when the activity meter, the electroencephalograph, or the camera is not available, information obtained according to subjective evaluations may be inputted to an input device including biological information obtainer 10, to use the information inputted as the biological information. For example, a sleep length, a time length taken for falling asleep, a time length taken for waking up, etc., which are subjectively evaluated by the user may be used as the biological information. In addition, for example, the number of pimples or acnes, etc., subjectively evaluated by the user may be used as the biological information.

The amount of sunlight the user is exposed to and the illuminance of light the user is exposed to each indicate the degree of alleviation of the user's symptoms of PMS. For example, when the illuminance exposure integrated value is high, the symptom of PMS becomes mild.

Severity determiner 40 determines the severity of PMS based on at least one of information items included in the biological information and at least one of information items included in the environmental information, the information items each being quantified according to a predetermined degree of influence on the severity of PMS. More specifically, severity determiner 40 determines the severity of PMS by adding together the information items included in the quantified biological information and environmental information. Severity determiner 40 calculates severity, for example, as follows: a0×a value corresponding to a current phase of the menstrual cycle+a1×a value corresponding to a sleep length+a2×a value corresponding to a level of bad skin condition+a3×a value corresponding to an amount of activity during daytime+a4×a value corresponding to an illuminance exposure integrated value+ . . . . Severity determiner 40 determines the severity to be higher as the value of the calculated severity is higher. The value corresponding to the current phase of the menstrual cycle is, for example, high when the user is currently in the period of developing PMS, and low when the user is currently in the period of not developing PMS. The value corresponding to the sleep length is, for example, high when the sleep length of the user is short, and low when the sleep length of the user is long. The value corresponding to the level of bad skin condition is, for example, high when the number of pimples or acnes of the user is large, and low when the number of pimples or acnes of the user is small. The value corresponding to the amount of activity during daytime is, for example, high when the user's amount of activity during daytime is large (i.e., when the user's tiredness or fatigue is severe), and low when the user's amount of activity during daytime is small. The value corresponding to the illuminance exposure integrated value is, for example, high when the integrated value of the illuminance of light the user is exposed to is low, and low when the integrated value of illuminance of light the user is exposed to is high. Other information items included in the biological information are omitted in the above-described expression for calculating severity. However, various information items related to a biological body of the user which are included in the biological information are summed up to calculate the severity of PMS.

In addition, a0, a1, a2, . . . are coefficients associated one-to-one with the information items included in the biological information and the environmental information. The coefficients are predetermined according to the degree of influence on the severity of PMS, and each of the information items is weighted by, for example, a coefficient of a higher value, as the information has a higher degree of influence on the severity of PMS. For example, since the value corresponding to the current phase of the menstrual cycle is intrinsically an information item indicating whether or not a user is in a period of developing PMS (in other words, whether or not PMS is developed), the degree of influence on the severity of PMS of the value is high. In other words, a0 is a coefficient having a high value. Accordingly, when the value corresponding to the current phase of the menstrual cycle is small (when, for example, a user is currently in the period of not developing PMS), the severity to be calculated is likely to be small, and when the value corresponding to the current phase of the menstrual cycle is large (when, for example, a user is currently in the period of developing PMS), the severity to be calculated is likely to be large.

In this manner, severity determiner 40 determines the severity of PMS to be, for example, “no symptom”, “mild”, “moderate”, “severe” and “premenstrual dysphoric disorder (PMDD)”, in ascending order of the calculated value of the severity. Here, PMDD is a disorder which is severer than PMS.

In this manner, severity determiner 40 determines the severity of PMS based on the biological information and the environmental information.

Next, lighting controller 50 controls a lighting device based on the severity of PMS determined by severity determiner 40 (Step S13). More specifically, lighting controller 50 controls luminance, a color temperature, and a light emission duration of the lighting device, based on the severity of PMS determined by severity determiner 40. For example, lighting controller 50 controls luminance such that a higher the severity is, the higher the luminance becomes, the higher the color temperature becomes, and the longer the light emission duration becomes. For example, when severity determiner 40 determines that the severity of PMS is large (PMDD, for example), lighting controller 50 emits light having a high level of luminance and a high color temperature for a long period of time. When severity determiner 40 determines that the severity of PMS is small (mild, for example), lighting controller 50 emits light having a low level of luminance and a low color temperature for a short period of time.

[Application Example of Lighting Control System]

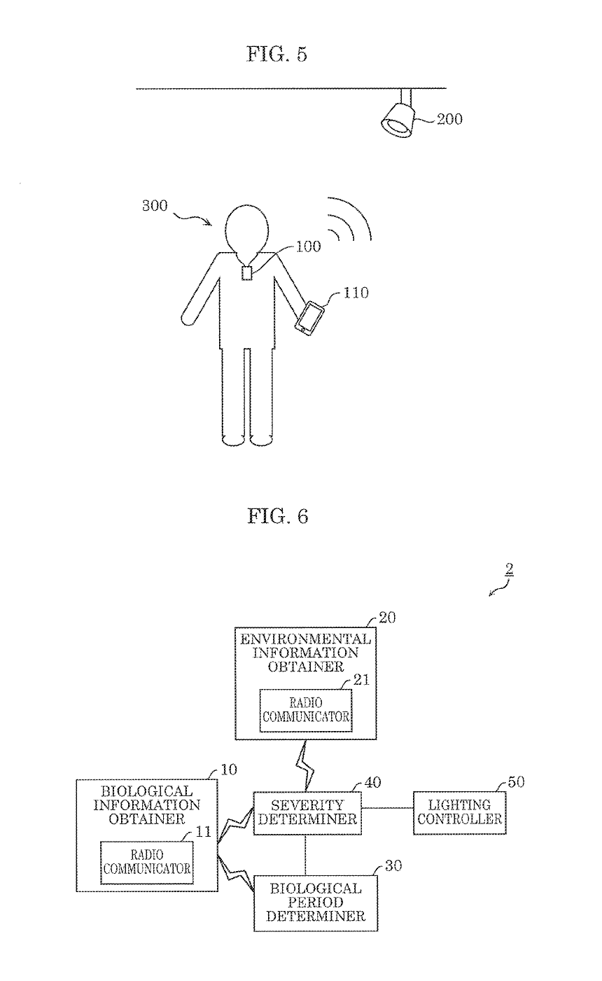

Next, an application example of lighting control system 1 will be described with reference to FIG. 5.

FIG. 5 is a diagram illustrating an application example of lighting control system 1 according to Embodiment 1.

As illustrated in FIG. 5, user 300 wears wearable terminal 100 and holds smartphone 110. In addition, user 300 is about to receive illuminating light from lighting device 200.

In this application example, environmental information obtainer 20 is, for example, wearable terminal 100. Biological information obtainer 10 is, for example, smartphone 110. Severity determiner 40 and lighting controller 50 are included, for example, in lighting device 200. Wearable terminal 100 and smartphone 110 wirelessly communicate with lighting device 200.

Suppose that, for example, user 300 has taken an outdoor walk in a clear morning. Environmental information obtainer 20 wirelessly transmits, for example, an illuminance exposure integrated value of the morning as the environmental information, to severity determiner 40. Furthermore, smartphone 110 wirelessly transmits, for example, the biological information inputted by user 300, to severity determiner 40. In lighting device 200, severity determiner 40 determines the severity of PMS based on the biological information and the environmental information which have been received, and lighting controller 50 controls lighting device 200 based on the severity of PMS determined by severity determiner 40.

It should be noted that, although it has been described that environmental information obtainer 20 wirelessly transmits, for example, an illuminance exposure integrated value of the morning as the environmental information, to severity determiner 40, the environmental information to be transmitted is not limited to this example. For example, environmental information obtainer 20 may transmit an illuminance exposure integrated value of the previous day as the environmental information, to severity determiner 40. More specifically, severity determiner 40 may determine the severity of PMS based on, for example, an illuminance exposure integrated value of a predetermined period of time (for example, three hours in the morning, etc.) as the environmental information, or based on, for example, an illuminance exposure integrated value of a predetermined date (for example, the previous day).

Moreover, although lighting device 200 is a spotlight according to the present application example as illustrated in FIG. 5, lighting device 200 may be a ceiling light, a stand light, etc. In addition, lighting device 200 may be a lighting device attached to a dresser or the like.

[Advantageous Effects, Etc.]

During the period of developing PMS, the degree of the symptoms of PMS varies between severe and mild, depending on the amount of activity of a user, a period of time during which a user is exposed to sunlight or illuminating light, etc. However, conventionally, there has not been environmental control according to variation of the severity of PMS. Moreover, conventional environmental control, for example, targets only the temperature environment in a bed, and does not target a lighting environment.

In view of the above, lighting control system 1 according to the present embodiment includes biological information obtainer 10 which obtains biological information related to a biological body of user 300, and environmental information obtainer 20 which obtains environmental information related to a surrounding environment of user 300. In addition, lighting control system 1 includes severity determiner 40 which determines a severity of PMS based on the biological information obtained by biological information obtainer 10 and the environmental information obtained by environmental information obtainer 20, and lighting controller 50 which controls lighting device 200 based on the severity determined by severity determiner 40.

For example, lighting control system 1 includes: a computer; and a non-transitory computer readable medium storing a program. The computer executes the program to obtain biological information related to a biological body of user 300, from a sensor, and information related to a surrounding environment of user 300, from an illuminance meter. The computer determines a severity of premenstrual syndrome (PMS) based on the biological information and the environmental information, and controls lighting device 200 based on the severity.

It should be noted that lighting control system 1 may include: lighting device 200; a sensor that obtains biological information related to a biological body of user 300; an illuminance meter that obtains environmental information relating to at least one of an amount of sunlight user 300 is exposed to and an illuminance of light user 300 is exposed to; a computer; and a non-transitory computer readable medium storing a program. In addition, the computer, by executing the program, may determine a severity of premenstrual syndrome (PMS) based on the biological information and the environmental information; and control lighting device 200 based on the severity.

With the above-described configuration, since the symptoms of PMS vary according to, for example, a sleep length or an amount of activity during daytime, and are alleviated by receiving sunlight or illuminating light, it is possible to determine the severity of PMS by obtaining information related to, for example, the sleep length or the amount of activity during daytime as the biological information, and obtaining information related to, for example, the illuminance of light the user is exposed to as the environmental information. Lighting device 200 is controlled so as to alleviate the symptoms of PMS, based on the severity of PMS. More specifically, it is possible to alleviate the symptoms of PMS in response to the severity of PMS which varies according to activity contents of user 300, a time length during which user 300 is exposed to light, etc.

Lighting controller 50 controls luminance, a color temperature, and a light emission duration of lighting device 200, based on the severity. More specifically, lighting controller 50 controls luminance such that a higher the severity is, the higher the luminance of lighting device 200 becomes. Lighting controller 50 controls luminance such that a higher the severity is, the higher the color temperature of lighting device 200 becomes. Lighting controller 50 controls luminance such that a higher the severity is, the longer the light emission duration of lighting device 200 becomes.

In this manner, since the symptoms of PMS are alleviated by receiving light having a high level of luminance and a high color temperature for a long period of time, it is possible to efficiently alleviate the symptoms of PMS by controlling luminance, a color temperature, and a light emission duration of lighting device 200, based on the severity of PMS.

The biological information includes at least one of information items which indicate a basal body temperature, an amount of luteinizing hormone secretion, an amount of luteohormone secretion, an amount of estrogenic hormone secretion, an amount of activity, blinking, electrodermal activity, brain waves, a skin condition, a date of menstruation onset, and the number of days of a menstrual cycle of user 300.

With this, it is possible to determine the severity of PMS, based on a menstrual cycle, sleep, tiredness, drowsiness, etc., of user 300.

The environmental information includes at least one of information items which indicate an amount of sunlight user 300 is exposed to and an illuminance of light user 300 is exposed to.

With this, it is possible to determine the severity of PMS, based on the amount of sunlight user 300 is exposed to or the illuminance of light user 300 is exposed to.

Severity determiner 40 determines the severity based on at least one of information items included in the biological information and at least one of information items included in the environmental information, each of the information items being quantified according to a predetermined degree of influence on the severity.

In this manner, it is possible to determine the severity of PMS by integrating a plurality of information items with different degrees of influence on the severity of PMS, which are included in the biological information and the environmental information.

Biological information obtainer 10 and environmental information obtainer 20 include radio communicator 11 and radio communicator 21, respectively, for wirelessly transmitting the biological information and the environmental information.

In this manner, it is possible for user 300 to transmit the biological information and the environmental information, using smartphone 110, wearable terminal 100, etc., which are portable.

### Embodiment 2

The following describes Embodiment 2 with reference to FIG. 6 and FIG. 7.

Lighting control system 2 according to the present embodiment is different from lighting control system 1 according to Embodiment 1 in that lighting control system 2 includes biological period determiner 30, and is the same as lighting control system 1 according to Embodiment 1 other than the inclusion of biological period determiner 30. Accordingly, detailed description will be omitted, and the following describes mainly biological period determiner 30.

FIG. 6 is a diagram illustrating a configuration of lighting control system 2 according to Embodiment 2. As illustrated in FIG. 6, lighting control system 2 has the configuration of lighting control system 1 to which biological period determiner 30 is added. Biological period determiner 30 is included, for example, in lighting device 200 illustrated in FIG. 5, and wirelessly communicates with radio communicator 11.

FIG. 7 is a flowchart illustrating an operation of lighting control system 2 according to Embodiment 2. As illustrated in FIG. 7, the operation of lighting control system 2 includes determination of Step S21 added between Step S11 and Step S12 performed by lighting control system 1.

Biological period determiner 30 determines whether or not a user is in a period of developing PMS, based on the biological information obtained by biological information obtainer 10 (Step S21). For example, biological period determiner 30 determines whether or not a user is currently in a period of developing PMS, based on a basal body temperature, an amount of luteinizing hormone secretion, an amount of luteohormone secretion, an amount of estrogenic hormone secretion, a date of menstruation onset, the number of days of a menstrual cycle, etc., which are obtained by biological information obtainer 10.

When biological period determiner 30 determines that the user is in the period of developing PMS (Yes, in Step S21), severity determiner 40 determines the severity of PMS.

When biological period determiner 30 determines that the user is in the period of not developing PMS (No, in Step S21), severity determiner 40 skips determining of the severity of PMS. More specifically, when biological period determiner 30 determines that the user is in the period of not developing PMS, severity determiner 40 does not determine the severity of PMS, and lighting controller 50 does not control a lighting device.

As described above, lighting control system 2 further includes biological period determiner 30 which determines whether or not the user is in a period of developing PMS, based on the biological information obtained by biological information obtainer 10, and severity determiner 40 determines the severity when biological period determiner 30 determines that the user is in the period of developing PMS.

In this manner, determination of severity is not carried out and a lighting device is not controlled in the period of not developing PMS. This means that determination of severity and control on a lighting device are carried out only in the period of developing PMS, and thus it is possible to reduce power used by lighting control system 2. It should be noted that control on a lighting device different from the control on the lighting device carried out in the period of developing PMS may be carried out in the period of not developing PMS.

### Other Embodiment

Although a lighting control system according to the embodiments has been described thus far, the present disclosure is not limited to the above-described embodiments.

For example, although the environmental information obtained by environmental information obtainer 20 is used for determination performed by severity determiner 40 according to the above-described embodiments, the present disclosure is not limited to this example. For example, the environmental information obtained by environmental information obtainer 20 may be used by lighting controller 50 for controlling a lighting device, instead of determination performed by severity determiner 40.

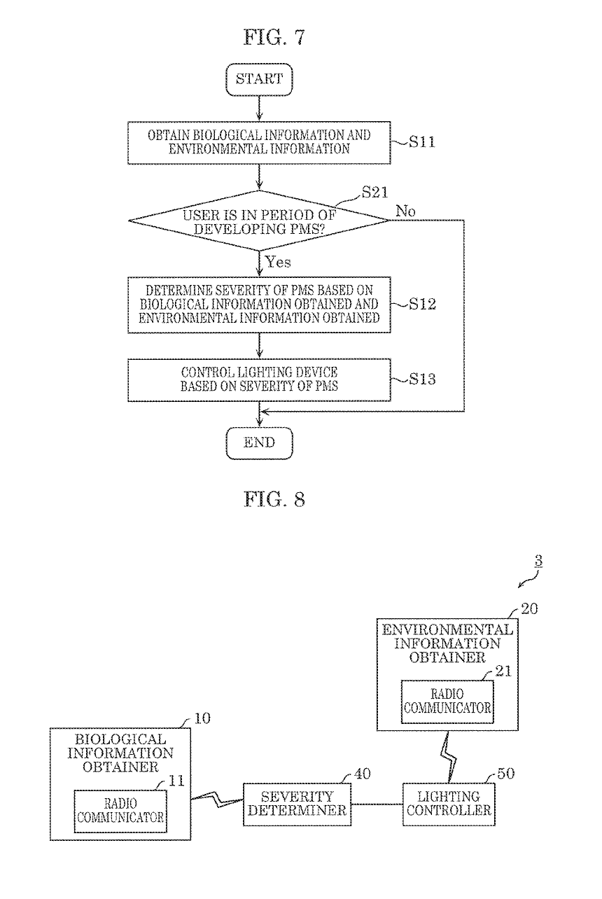

FIG. 8 is a diagram illustrating a configuration of lighting control system 3 according to another embodiment.

Severity determiner 40 communicates with biological information obtainer 10 and environmental information obtainer 20 according to the above-described embodiments. However, severity determiner 40 in this example does not communicate with environmental information obtainer 20. Lighting controller 50 communicates with severity determiner 40 and environmental information obtainer 20. In other words, as in lighting control system 3, the severity of PMS may be determined based on the biological information obtained by biological information obtainer 10, and a lighting device may be controlled based on the environmental information and the information related to the severity of PMS based on the biological information.

As described above, lighting control system 3 according to the other embodiment includes biological information obtainer 10 which obtains biological information related to a biological body of a user, and environmental information obtainer 20 which obtains environmental information related to a surrounding environment of the user. In addition, lighting control system 3 includes severity determiner 40 which determines the severity of PMS based on the biological information obtained by biological information obtainer 10, and lighting controller 50 which controls a lighting device based on the environmental information obtained by environmental information obtainer 20 and the severity determined by severity determiner 40.

In addition, for example, although lighting controller 50 controls luminance, a color temperature, and a light emission duration of a lighting device according to the above-described embodiments, the present disclosure is not limited to this example. For example, lighting controller 50 may control a time zone of emitting light. In this manner, a circadian rhythm of the user is controlled, and not only alleviation of the symptoms of PMS but also alleviation of the symptoms of depression, for example, can be expected.

In addition, for example, although biological information obtainer 10 is smartphone 110 according to the above-described embodiments, the present disclosure is not limited to this example. For example, biological information obtainer 10 may be an activity meter, an electroencephalograph, a camera, a PC, a wearable terminal, etc.

In addition, for example, although environmental information obtainer 20 is wearable terminal 100 according to the above-described embodiments, the present disclosure is not limited to this example. For example, environmental information obtainer 20 may be a PC, a smartphone, etc.

Furthermore, for example, although biological period determiner 30, severity determiner 40, and lighting controller 50 are included in lighting device 200, the present disclosure is not limited to this example. For example, biological period determiner 30, severity determiner 40, and lighting controller 50 may be included in a PC, a smartphone, etc.

These general and specific aspects of the present disclosure may be implemented using a system, a method, an integrated circuit, a computer program, or a computer-readable recording medium such as a compact disc read only memory (CD-ROM), or any combination of systems, methods, integrated circuits, computer programs, or computer-readable recording media.

It should be noted that the present disclosure also includes other forms in which various modifications apparent to those skilled in the art are applied to the embodiments or forms in which structural components and functions in the embodiments are arbitrarily combined within the scope of the present disclosure.

While the foregoing has described one or more embodiments and/or other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present teachings.

What is claimed is:

1. A lighting control system, comprising:
a biological information obtainer which obtains biological information related to a biological body of a user;
an environmental information obtainer which obtains environmental information related to a surrounding environment of the user;
a severity determiner which determines a severity of premenstrual syndrome (PMS) based on the biological information obtained by the biological information obtainer and the environmental information obtained by the environmental information obtainer; and
a lighting controller which controls a lighting device based on the severity determined by the severity determiner.

2. The lighting control system according to claim 1, further comprising
a biological period determiner which determines whether or not the user is in a period of developing PMS, based on the biological information obtained by the biological information obtainer, wherein
the severity determiner determines the severity when the biological period determiner determines that the user is in the period of developing PMS.

3. The lighting control system according to claim 1, wherein
the lighting controller controls luminance, a color temperature, and a light emission duration of the lighting device, based on the severity.

4. The lighting control system according to claim 3, wherein
the lighting controller controls luminance such that a higher the severity is, the higher the luminance becomes.

5. The lighting control system according to claim 3, wherein
the lighting controller controls luminance such that a higher the severity is, the higher the color temperature becomes.

6. The lighting control system according to claim 3, wherein
the lighting controller controls luminance such that a higher the severity is, the longer the light emission duration becomes.

7. The lighting control system according to claim 1, wherein
the biological information includes at least one of information items which indicate a basal body temperature, an amount of luteinizing hormone secretion, an amount of luteohormone secretion, an amount of estrogenic hormone secretion, an amount of activity, blinking, electrodermal activity, brain waves, a skin condition, a date of menstruation onset, and the number of days of a menstrual cycle of the user.

8. The lighting control system according to claim 1, wherein
the environmental information includes at least one of information items which indicate an amount of sunlight the user is exposed to and an illuminance of light the user is exposed to.

9. The lighting control system according to claim 1, wherein
the severity determiner determines the severity based on at least one of information items included in the biological information and at least one of information items included in the environmental information, the information items each being quantified according to a predetermined degree of influence on the severity.

10. The lighting control system according to claim 1, wherein
the biological information obtainer and the environmental information obtainer each include a radio communicator for wirelessly transmitting a corresponding one of the biological information and the environmental information.

11. A lighting control system, comprising:
a biological information obtainer which obtains biological information related to a biological body of a user;
an environmental information obtainer which obtains environmental information related to a surrounding environment of the user;
a severity determiner which determines a severity of premenstrual syndrome (PMS) based on the biological information obtained by the biological information obtainer; and
a lighting controller which controls a lighting device based on the environmental information obtained by the environmental information obtainer and the severity determined by the severity determiner.

12. A lighting control system, comprising:
a computer; and
a non-transitory computer readable medium storing a program,
wherein the program, when executed by the computer, causes the computer to:
obtain biological information related to a biological body of a user, from a sensor; obtain environmental information related to a surrounding environment of the user, from an illuminance meter; determine a severity of premenstrual syndrome (PMS) based on the biological information and the environmental information; and
control a lighting device based on the severity.

13. A lighting control system, comprising:
a lighting device;
a sensor that obtains biological information related to a biological body of a user;
an illuminance meter that obtains environmental information relating to at least one of an amount of sunlight the user is exposed to and an illuminance of light the user is exposed to;
a computer; and
a non-transitory computer readable medium storing a program,
wherein the program, when executed by the computer, causes the computer to:
determine a severity of premenstrual syndrome (PMS) based on the biological information and the environmental information; and
control the lighting device based on the severity.
